(19) United States
(12) Patent Application Publication
(10) Pub. No.: US 2014/0171751 A1
(43) Pub. Date: Jun. 19, 2014

(54) ELECTRONIC BIO MONITORING PATCH

(71) Applicants: Robert L. Sankman, Phoenix, AZ (US); Ian A. Young, Portland, OR (US); Johanna M. Swan, Scottsdale, AZ (US); Marko Radosavljevic, Beaverton, OR (US)

(72) Inventors: Robert L. Sankman, Phoenix, AZ (US); Ian A. Young, Portland, OR (US); Johanna M. Swan, Scottsdale, AZ (US); Marko Radosavljevic, Beaverton, OR (US)

(21) Appl. No.: 13/719,360

(22) Filed: Dec. 19, 2012

(51) Int. Cl. A61B 5/0205; A61B 5/024; A61B 5/145; A61B 5/021; A61B 5/0488; A61N 5/00; A61B 5/01; A61B 5/0245; A61N 1/36; A61N 5/06; A61H 99/00; A61B 5/00; A61B 5/0476

(52) U.S. Cl. CPC A61B 5/02055; A61B 5/00; A61B 5/0002; A61B 5/02438; A61B 5/14532; A61B 5/14542; A61B 5/021; A61B 5/0488; A61B 5/0476; A61B 5/01; A61B 5/0205; A61B 5/0245; A61B 5/0004; A61B 5/0006; A61B 5/0008; A61B 5/6833; A61N 1/36014; A61B 5/4839; A61N 5/06; A61H 99/00; A61N 5/00
USPC 600/301; 600/300; 600/508; 600/365; 600/364; 600/485; 600/546; 600/544; 600/549; 600/483

(57) ABSTRACT

Described herein are technologies related to a wireless electronic vital sign monitoring of a person. More particularly, detecting vital signs and processing of the detected vital signs using a bio monitoring patch.

### BACKGROUND

A conventional vital signal monitoring of a person typically requires a strap or a garment to hold a device to a specific area where the vital sign is to be measured. For example, when measuring blood pressure of the person, blood pressure equipment is strapped to an arm of the person. Due to the bulkiness and relatively large size of electronic circuitry associated with this portable medical device (i.e., blood pressure equipment), the electronic circuitry is typically external to the equipment itself. For example, processing circuits are implemented through an external computer that is attached to the blood pressure equipment.

Furthermore, the conventional portable medical devices are often utilized in multiple patients such that, a careful sterilization of the medical devices is observed every time that the portable medical device is used. Additionally, the weight and geometry of rigid containers enclosing the medical devices are not well-suited for flexibility during treatment of the person in an operating room where a number of medical equipment may be attached to the body of the person.

### BRIEF DESCRIPTION OF THE DRAWINGS

FIG. 1 illustrates an example scenario that shows different situations of utilizing a wearable bio monitoring patch to transmit data (e.g., health information) to a wireless device.

FIG. 2 illustrates an example top view of a system that implements a bio monitoring patch.

FIG. 3 illustrates an example underside view of a bio monitoring patch.

FIG. 4 illustrates an example system of a wireless device in accordance with present disclosure.

FIG. 5 illustrates an example flowchart of an example method of implementing electronic vital sign monitoring system on chip (SOC) device.

### DETAILED DESCRIPTION

Described herein is a technology for a wireless electronic vital sign monitoring of a person. More particularly, a method of detecting vital signs and processing of the detected vital signs is described.

For example, a bio monitoring patch—at detecting mode—is configured to detect and store vital signs such as body temperature, heartbeat rate, and the like of a patient. In this example, a system on a chip (SOC) microprocessor in the bio monitoring patch is configured to utilize the stored vital signs as variables to a pre-configured program such as, generating a summary of the patient's vital signs in the last twelve hours. In another example, the SOC microprocessor may be configured to control periods of detection by multiple stimulators, stimulator contacts, or sensors in the bio monitoring patch. Such stimulators can deliver medicine, electrical, optical, radio frequency (RF) or mechanical energy to the body.

As an example of current implementations herein, the bio monitoring patch may be configured to generate electrical pulses to stimulate muscles of the patient. In this example, the (SOC) microprocessor may be configured to control the generation of the electrical pulses that are used to treat or strengthen the muscles of the patient. In another example, the SOC microprocessor may be configured to control other machines that may be utilized in giving health care to the patient. For example, if an intravenous fluid (IV) is injected through a machine, then the SOC microprocessor may be configured to control wirelessly the machine based on detected vital signs of the patient.

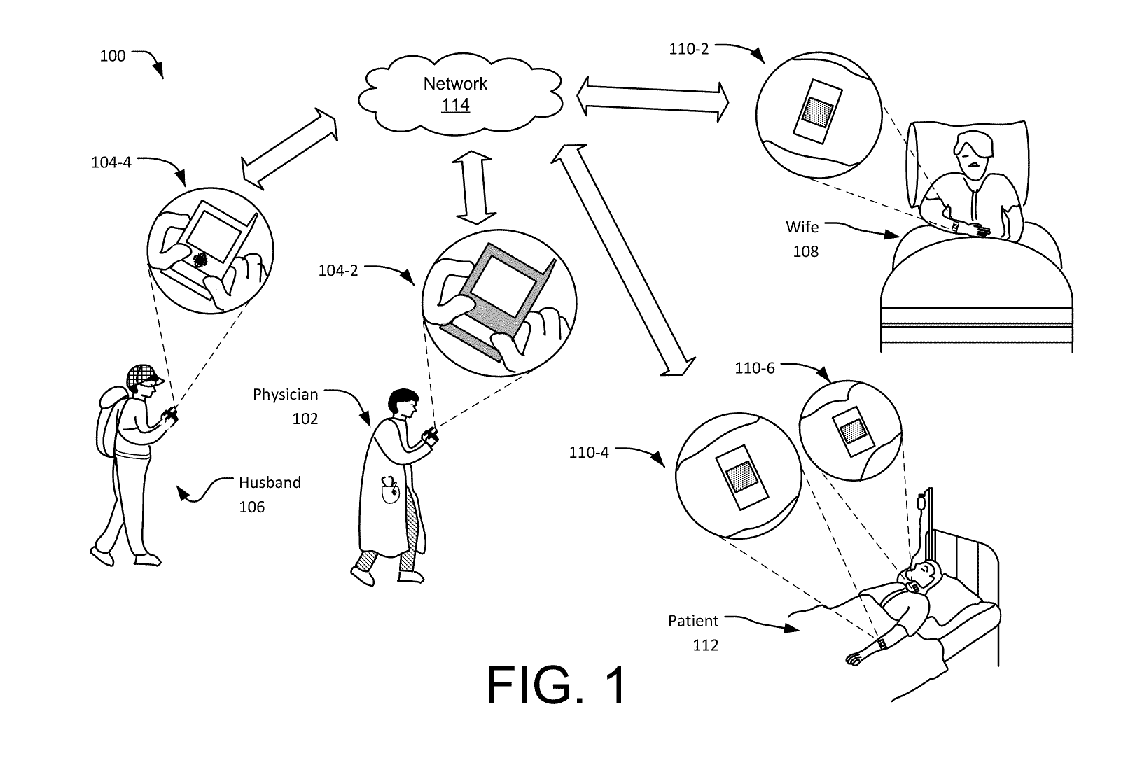

FIG. 1 illustrates a scenario 100 that shows different situations of utilizing a wearable bio monitoring patch to transmit data (e.g., health information) to a wireless device. As shown, scenario 100 depicts a physician 102 holding a wireless device 104-2, a husband 106 holding a wireless device 104-4, a wife 108 wearing a bio monitoring patch 110-2, and a patient 112 wearing bio monitoring patches 110-4 and 110-6. Furthermore, scenario 100 shows a network 114 that is utilized to establish wire/wireless communications between the wireless device 104 and the bio monitoring patch 106.

Scenario 100 depicts an example implementation of technology described herein. For example, the husband 106 is a person who works away from home and would like to check on health information updates of his wife (i.e., wife 108) who is currently at home and lying in bed. In this example, the husband 106 utilizes his wireless device 104-4 that is configured to receive the health information from the bio monitoring patch 110-2 that the wife 108 is wearing.

As an example of present implementation, the bio monitoring patch 110-2 above may be configured to detect vital signs such as, a current body temperature, blood pressure, blood sugar, and other body measurements of the wife 108. In this example, the bio monitoring patch 110-2 may be configured to process the detected vital signs and transmit a summary of the vital signs or body measurements to the wireless device 104-4 of the husband 106. In another example, the wife 108 may be at a grocery store and the husband 106 may check the health information updates of the wife 108 through the wireless device 104-4. In other implementations, stimulators, stimulator contacts, or sensors in the bio monitoring patch 110-2 may deliver medicine, electrical, optical, radio frequency (RF) or mechanical energy to the body.

In another example, the physician 102 is a medical doctor who is physically outside of a hospital (not shown) where the patient 112 is currently confined. In this example, the patient 112 is under the care of the physician 102 and the physician 102 utilizes his wireless device 104-2 to get health information updates of the patient 112. Similar to the discussion above, the bio monitoring patches 110-4 and 110-6, which the patient 112 are wearing, may be configured to make body measurements or detect vital signs on the patient 112 and transmit the body measurements or detected vital signs to the wireless device 104-2.

With continuing reference to FIG. 1, the bio monitoring patch 110 includes a wireless epidermal electronic sensor system that contains a miniaturized microprocessor with multiple stimulators, stimulator contacts, or sensors in the bio monitoring patch. Such stimulators can deliver medicine, electrical, optical, radio frequency (RF) or mechanical energy to the body. For example, such stimulators or sensors may detect the vital signs such as different body measurements on a person (e.g., wife 108 or patient 112). For example, the bio monitoring patch 110 is made of semiconductor materials that include a memory in addition to the miniaturized stimulators, stimulator contacts, or sensors, microprocessor, and the like. In this example, the bio monitoring patch 110 may perform external (i.e., epidermal) body measurements, or the bio monitoring patch 110 may be embedded inside the body of the person (i.e., wife 108 or the patient 112) to be monitored. In another example, the bio monitoring patch 110 may be used to generate electrical pulses to stimulate muscles of the wife 108 or the patient 112.

As an example of the present implementation, a radio signal (not shown) such as a cellular signal, a wireless fidelity (Wi-Fi) signal, and the like, may be used by the physician 102 or the husband 106 to access the bio monitoring patch 110. For example, the physician 102 may receive the remote health information updates through the cellular signal by receiving a text messaging. In this example, the wireless device 104-2 may receive the text messaging through the cellular signal and not through the network 114.

As depicted, the wireless device 104 may include, but not limited to, a mobile phone, a cellular phone, a smartphone, a personal digital assistant, a tablet computer, a netbook, a notebook computer, a laptop computer, a multimedia playback device, a digital music player, a digital video player, a navigational device, a digital camera, and the like.

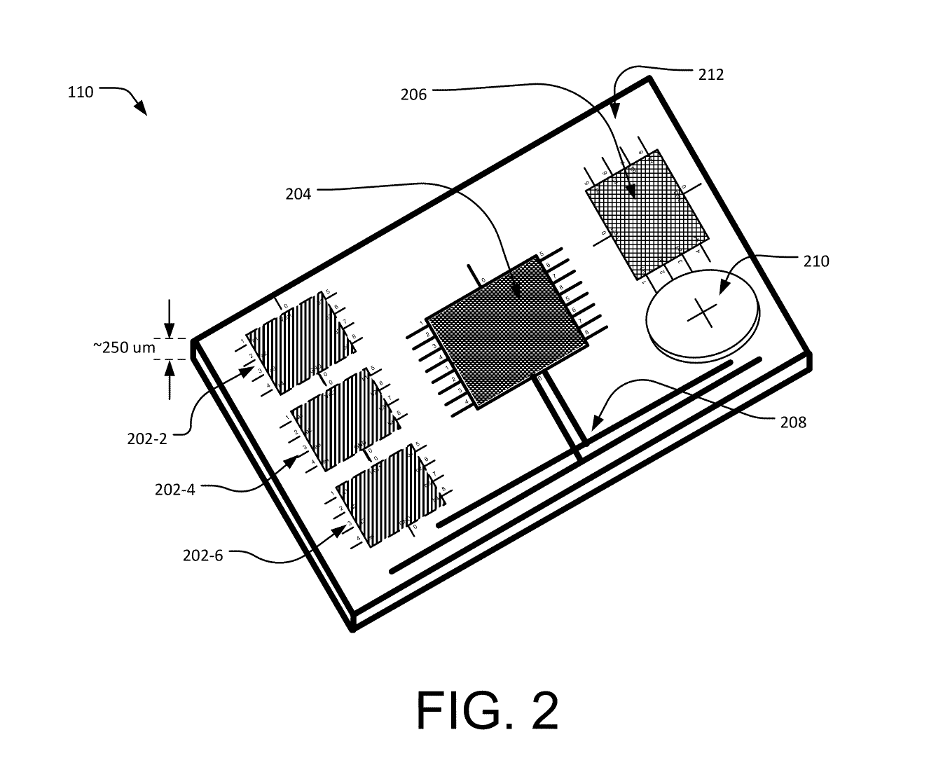

FIG. 2 illustrates an example top view of system 200 that implements the bio monitoring patch 110. System 200 shows multiple stimulators, stimulator contacts, or sensors 202, system on chip (SOC) microcontroller 204, a non-volatile memory (NVM) 206, an antenna 208, a battery 210 and a semiconductor material 212.

As an example of present implementation herein, the multiple stimulators, stimulator contacts, or sensors 202, at detecting mode, may be an epidermal body sensor that detects vital signs such as various body measurements. Typically, the body measurement is performed from outermost layer of a skin of the person being tested. The epidermal body sensor may detect body temperature, skin dryness, blood pressure, blood sugar, heartbeat rate, muscle activity, neurological activity, brain waves, and other physiological signals.

Upon detection of the vital signs above, the multiple stimulators, stimulator contacts, or sensors 202 may be configured to store the detected vital signs to the NVM 206. For example, the body temperature and the heart rate of the patient 112 for the last twelve hours are separately stored at the NVM 206. In this example, the SOC microcontroller 204 may be configured to interact with the multiple stimulators, stimulator contacts, or sensors 202. For example, the SOC microcontroller 204 may allow the multiple stimulators, stimulator contacts, or sensors 202 to detect the vital signals at a specific period of time only (e.g., every two hours in one day).

As an example of current implementations herein, the SOC microcontroller 204 may be configured to allow the multiple stimulators, stimulator contacts, or sensors 202-2, 202-4 and 202-6 to combine its separate sensors to detect the vital signs of the patient 112. For example, the SOC microcontroller 204 may be configured generate a summary that contains the measured body temperature for the last eight hours; heart rate average at the last ten hours; and muscle activity for the last twelve hours. In this example, the SOC microcontroller 204 may perform a configured calculation to generate the summary based on the vital sign measurements in the last twelve hours.

In another example, the SOC microcontroller 204, if not in detecting mode, may be configured to generate electrical pulses to stimulate muscles of the patient 112. For example, the physician 102 may prescribe staggered stimulation of different muscles of the patient 112 who is recovering from paralyzed state. In this example, the SOC microcontroller 204 may be configured or programmed to enable the multiple stimulators, stimulator contacts, or sensors 202 to generate the electrical signals at different body parts (e.g., neck, hand, foot) of the patient 112.

As an example of current implementations herein, the SOC microcontroller 204, if not in detecting mode, may be further configured to control other machine/s that may be used to give health care to the patient 112. For example, if injection of intravenous fluids (IV) is made through a machine with an electronic control, then the SOC microcontroller 204 may be configured to control the machine based upon the summary generated by the multiple stimulators, stimulator contacts, or sensors 202.

With continuing reference to FIG. 2, the antenna 208 may be a Wi-Fi antenna, an NFC antenna, a Bluetooth™ antenna or an RF antenna. For example, if there is no available network or cellular signals in a particular area where that patient 112 is located, then the physician 102 may utilize the NFC feature of the wireless device 104. In this example, the wireless device 104 is tapped into the bio monitoring patch 110 to establish wireless communications.

As another example of the antenna 208 implementation, the bio monitoring patch 110 may wirelessly communicate with the wireless device 104 using the Wi-Fi signal. For example, the antenna 208 is configured to operate at Wi-Fi signal frequency (e.g., 2.4 GHz band). In this example, the wireless device 104 may connect to the bio monitoring patch 110 through the Wi-Fi signal.

As shown, the bio monitoring patch 110 may be configured to be thin (e.g., less than two hundred fifty micrometers) and flexible so that the SOC microcontroller 204, the NVM 206 and the other components in the SOC device may not be damaged when bended.

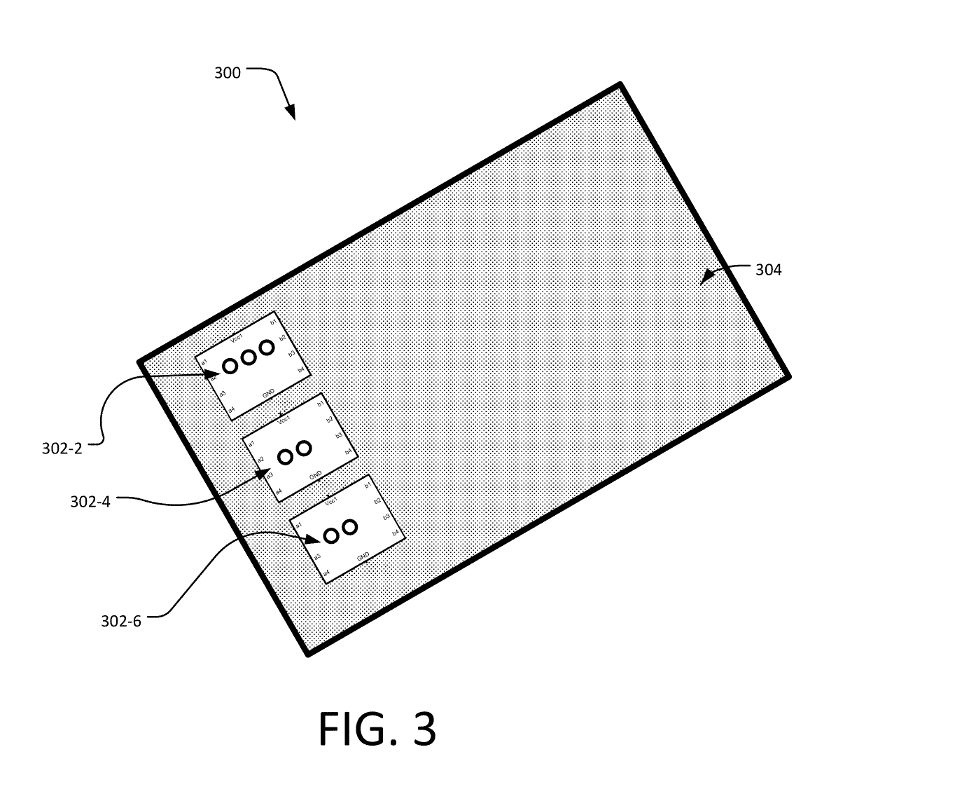

FIG. 3 illustrates an example underside view of the system 300 to implement the bio monitoring patch 110. The underside view shows contact points 302, and adhesive 304.

As an example of present implementation, contact point 302-2 is configured to detect body temperature of the patient 112. In this example, the contact point 302-2 may include a transducer that converts body heat into an electrical energy. The electrical energy may then be used to generate digital measurements of the body heat of the patient 112.

In another example, contact point 302-4 detects heartbeat rate of the patient 112. In this example, the heartbeat rate may generate an acoustical wave that is transformed into electrical signals by the contact points 302-4. The detected heartbeat rate and the body temperature as detected by the contact point 302-2 may be integrated together in producing a summary of the vital signs of the patient 112. For example, the SOC microcontroller 204 is configured to transmit an emergency signal if a certain combination of the detected heartbeat rate and body temperature is reached. In this example, the SOC microcontroller 204 may be configured to process both electrical signals that may be generated by the contact points 302-2 and 302-4.

In another example, contact point 302-6 is configured to detect amount of oxygen from the body of the patient 112. In this example, the SOC microcontroller 204 may be configured to relate the detected amount of oxygen with previous samples stored in the NVM 206. For example, an average of the detected amount of oxygen within last twenty four hours on the body of the patient 112 when combined with the current body temperature and heartbeat rate may signify to the physician 102 to prescribe oral medication to the patient 112. In this example, the SOC microcontroller 204 may transmit the information to the wireless device 104 of the physician 102.

As an example of current implementations herein, the contact point 302 may be utilized to deliver current or electrical pulses to the patient 112. For example, the electrical pulses are delivered through the skin of the patient 112. Since the skin is conductive by nature, the electrical pulses will reach and stimulate the muscles of the patient 112. The stimulation of the muscles is usually utilized to create strength in the muscles of the patient 112. In other implementations, contact point 302 may deliver medicine, optical, RF or mechanical energy.

With continuing reference to FIG. 3, the adhesive 304 may contain skin adhesive hydrogels to attach firmly the bio monitoring patch 110 to the patient 112. For example, the skin adhesive hydrogels may allow the contact points 302 to detect freely the vital signs of the patient 112 due to a very thin nature of the adhesive 304 that is utilized to attach the bio monitoring patch 110.

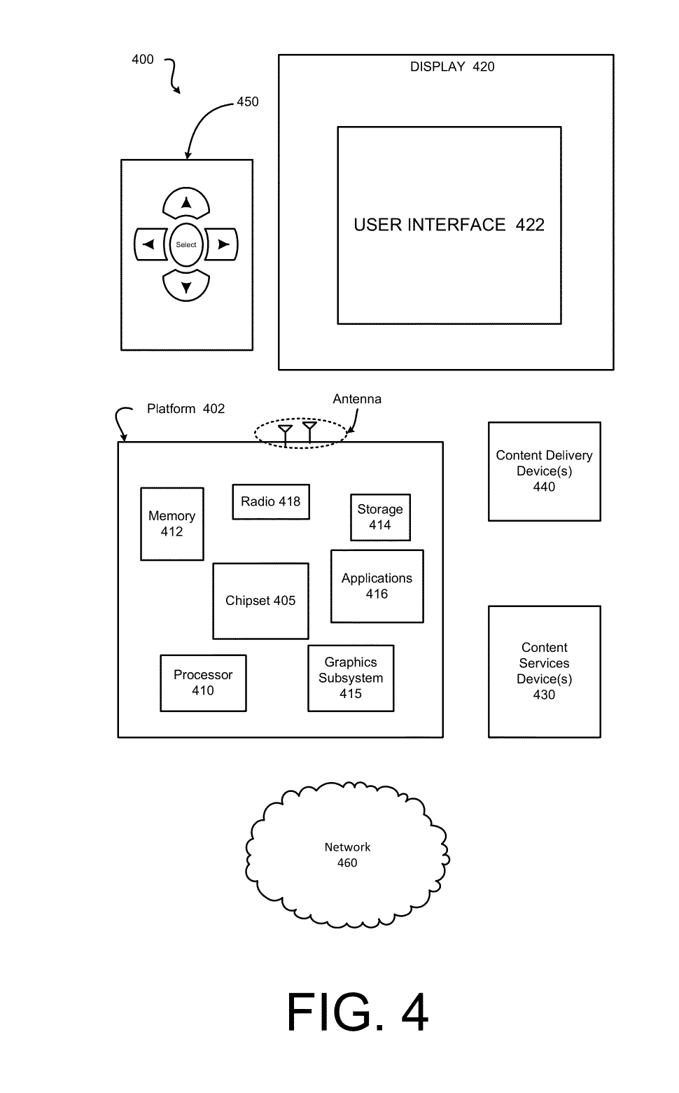

FIG. 4 illustrates an example system 400 of the wireless device 104 in accordance with present disclosure. In various implementations, system 400 may be a media system although system 400 is not limited to this context. For example, system 400 may be incorporated into a personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

In various implementations, system 400 includes a platform 402 coupled to a display 420. Platform 402 may receive content from a content device such as content services device(s) 430 or content delivery device(s) 440 or other similar content sources. A navigation controller 450 including one or more navigation features may be used to interact with, for example, platform 402 and/or display 420. Each of these components is described in greater detail below.

In various implementations, platform 402 may include any combination of a chipset 405, processor 410, memory 412, storage 414, graphics subsystem 415, applications 416 and/or radio 418. Chipset 405 may provide intercommunication among processor 410, memory 412, storage 414, graphics subsystem 415, applications 416 and/or radio 418. For example, chipset 405 may include a storage adapter (not depicted) capable of providing intercommunication with storage 414.

Processor 410 may be implemented as a Complex Instruction Set Computer (CISC) or Reduced Instruction Set Computer (RISC) processors, x86 instruction set compatible processors, multi-core, or any other microprocessor or central processing unit (CPU). In various implementations, processor 410 may be dual-core processor(s), dual-core mobile processor(s), and so forth that is coupled to the PIC as discussed in FIG. 2 above.

As an example of current implementations herein, the processor 410 is configured to control operations of the bandage patch 110. For example, the processor 410 is configured to include a software program that utilizes data from the NVM 206. In this example, the processing in the SOC microprocessor 204 is controlled by the software program in the processor 410 of the wireless device 104. To this end, the wireless device 104—in passive mode—is merely a wireless extension of the wireless device 104 through the network 114 or other radio signal. For example, the functions discussed in FIGS. 2 and 3 with regard to the SOC microprocessor 204 are directly performed by the physician 102 through the wireless device 104-2. For example, the detected vital signs in the last twenty four hours from the patient 112 are received and utilized as variables execution of the software program in the processor 410 of the system 400.

Memory 412 may be implemented as a volatile memory device such as, but not limited to, a Random Access Memory (RAM), Dynamic Random Access Memory (DRAM), or Static RAM (SRAM).

Storage 414 may be implemented as a non-volatile storage device such as, but not limited to, a magnetic disk drive, optical disk drive, tape drive, an internal storage device, an attached storage device, flash memory, battery backed-up SDRAM (synchronous DRAM), and/or a network accessible storage device. In various implementations, storage 414 may include technology to increase the storage performance enhanced protection for valuable digital media when multiple hard drives are included, for example.

As an example of current implementations herein, the storage 414 stores received data from the bandage patch 110. In this example, the data is utilized by the processor 410 to direct and control operations of the bandage patch 110.

Graphics subsystem 415 may perform processing of images such as still or video for display. Graphics subsystem 415 may be a graphics processing unit (GPU) or a visual processing unit (VPU), for example. An analog or digital interface may be used to communicatively couple graphics subsystem 415 and display 420. For example, the interface may be any of a High-Definition Multimedia Interface, DisplayPort, wireless HDMI, and/or wireless HD compliant techniques. Graphics subsystem 415 may be integrated into processor 410 or chipset 405. In some implementations, graphics subsystem 415 may be a stand-alone card communicatively coupled to chipset 405.

The graphics and/or video processing techniques described herein may be implemented in various hardware architectures. For example, graphics and/or video functionality may be integrated within a chipset. Alternatively, a discrete graphics and/or video processor may be used. As still another implementation, the graphics and/or video functions may be provided by a general purpose processor, including a multi-core processor. In further embodiments, the functions may be implemented in a consumer electronics device.

Radio 418 may include one or more radios capable of transmitting and receiving signals using various suitable wireless communications techniques. Such techniques may involve communications across one or more wireless networks. Example wireless networks include (but are not limited to) wireless local area networks (WLANs), wireless personal area networks (WPANs), wireless metropolitan area network (WMANs), cellular networks, and satellite networks. In communicating across such networks, radio 418 may operate in accordance with one or more applicable standards in any version.

In various implementations, display 420 may include any television type monitor or display. Display 420 may include, for example, a computer display screen, touch screen display, video monitor, television-like device, and/or a television. Display 420 may be digital and/or analog. In various implementations, display 420 may be a holographic display. Also, display 420 may be a transparent surface that may receive a visual projection. Such projections may convey various forms of information, images, and/or objects. For example, such projections may be a visual overlay for a mobile augmented reality (MAR) application. Under the control of one or more software applications 416, platform 402 may display user interface 422 on display 420.

In various implementations, content services device(s) 430 may be hosted by any national, international and/or independent service and thus accessible to platform 402 via the Internet, for example. Content services device(s) 430 may be coupled to platform 402 and/or to display 420. Platform 402 and/or content services device(s) 430 may be coupled to a network 460 to communicate (e.g., send and/or receive) media information to and from network 460. Content delivery device(s) 440 also may be coupled to platform 402 and/or to display 420.

In various implementations, content services device(s) 430 may include a cable television box, personal computer, network, telephone, Internet enabled devices or appliance capable of delivering digital information and/or content, and any other similar device capable of unidirectionally or bidirectionally communicating content between content providers and platform 402 and/display 420, via network 460 or directly. It will be appreciated that the content may be communicated unidirectionally and/or bidirectionally to and from any one of the components in system 400 and a content provider via network 460. Examples of content may include any media information including, for example, video, music, medical and gaming information, and so forth.

Content services device(s) 430 may receive content such as cable television programming including media information, digital information, and/or other content. Examples of content providers may include any cable or satellite television or radio or Internet content providers. The provided examples are not meant to limit implementations in accordance with the present disclosure in any way.

In various implementations, platform 402 may receive control signals from navigation controller 450 having one or more navigation features. The navigation features of controller 450 may be used to interact with user interface 422, for example. In embodiments, navigation controller 450 may be a pointing device that may be a computer hardware component (specifically, a human interface device) that allows a user to input spatial (e.g., continuous and multi-dimensional) data into a computer. Many systems such as graphical user interfaces (GUI), and televisions and monitors allow the user to control and provide data to the computer or television using physical gestures.

Movements of the navigation features of controller 450 may be replicated on a display (e.g., display 420) by movements of a pointer, cursor, focus ring, or other visual indicators displayed on the display. For example, under the control of software applications 416, the navigation features located on navigation controller 450 may be mapped to virtual navigation features displayed on user interface 422, for example. In embodiments, controller 450 may not be a separate component but may be integrated into platform 402 and/or display 420. The present disclosure, however, is not limited to the elements or in the context shown or described herein.

In various implementations, drivers (not shown) may include technology to enable users to instantly turn on and off platform 402 like a television with the touch of a button after initial boot-up, when enabled, for example. Program logic may allow platform 402 to stream content to media adaptors or other content services device(s) 430 or content delivery device(s) 440 even when the platform is turned “off.” In addition, chipset 405 may include hardware and/or software support for 5.1 surround sound audio and/or high definition 7.1 surround sound audio, for example. Drivers may include a graphics driver for integrated graphics platforms. In embodiments, the graphics driver may comprise a peripheral component interconnect (PCI) Express graphics card.

In various implementations, any one or more of the components shown in system 400 may be integrated. For example, platform 402 and content services device(s) 430 may be integrated, or platform 402 and content delivery device(s) 440 may be integrated, or platform 402, content services device(s) 430, and content delivery device(s) 440 may be integrated, for example. In various embodiments, platform 402 and display 420 may be an integrated unit. Display 420 and content service device(s) 430 may be integrated, or display 420 and content delivery device(s) 440 may be integrated, for example. These examples are not meant to limit the present disclosure.

In various embodiments, system 400 may be implemented as a wireless system, a wired system, or a combination of both. When implemented as a wireless system, system 400 may include components and interfaces suitable for communicating over a wireless shared media, such as one or more antennas, transmitters, receivers, transceivers, amplifiers, filters, control logic, and so forth. An example of wireless shared media may include portions of a wireless spectrum, such as the RF spectrum and so forth. When implemented as a wired system, system 400 may include components and interfaces suitable for communicating over wired communications media, such as input/output (I/O) adapters, physical connectors to connect the I/O adapter with a corresponding wired communications medium, a network interface card (NIC), disc controller, video controller, audio controller, and the like. Examples of wired communications media may include a wire, cable, metal leads, printed circuit board (PCB), backplane, switch fabric, semiconductor material, twisted-pair wire, co-axial cable, fiber optics, and so forth.

Platform 402 may establish one or more logical or physical channels to communicate information. The information may include media information and control information. Media information may refer to any data representing content meant for a user. Examples of content may include, for example, data from a voice conversation, videoconference, streaming video, electronic mail (“email”) message, voice mail message, alphanumeric symbols, graphics, image, video, text and so forth. Data from a voice conversation may be, for example, speech information, silence periods, background noise, comfort noise, tones and so forth. Control information may refer to any data representing commands, instructions or control words meant for an automated system. For example, control information may be used to route media information through a system, or instruct a node to process the media information in a predetermined manner. The embodiments, however, are not limited to the elements or in the context shown or described in FIG. 4.

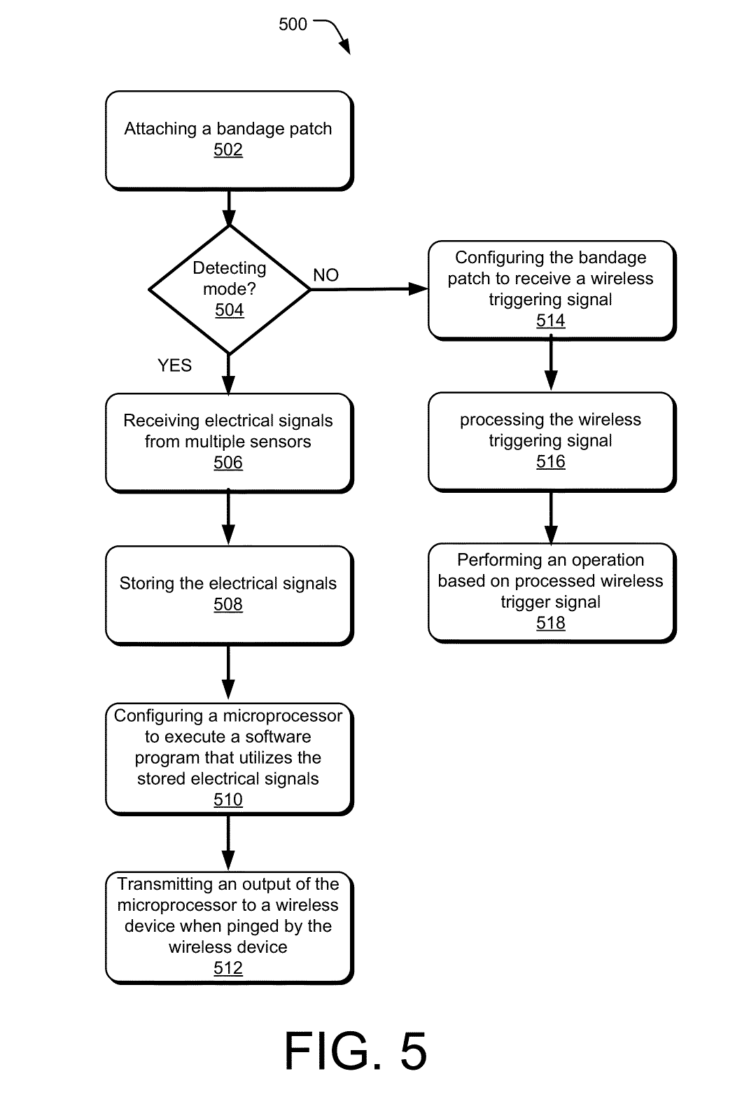

FIG. 5 shows an example process flowchart 500 illustrating an example method of implementing electronic vital sign monitoring SOC device. The order in which the method is described is not intended to be construed as a limitation, and any number of the described method blocks can be combined in any order to implement the method, or alternate method. Additionally, individual blocks may be deleted from the method without departing from the spirit and scope of the subject matter described herein. Furthermore, the method may be implemented in any suitable hardware, software, firmware, or a combination thereof, without departing from the scope of the invention.

At block 502, attaching a bio monitoring patch to a person to be monitored is performed. For example, the bio monitoring patch (e.g., bio monitoring patch 110) is attached to different body parts (e.g., hand, neck, head, etc.) of a person (e.g., patient 112). In this example, the bio monitoring patch 110 is correspondingly placed at a location in the body parts according to purpose of utilizing the bio monitoring patch 110. For example, if the bio monitoring patch 110 is utilized to detect heartbeat rate of the patient 112, then the bio monitoring patch 110 is placed in a wrist, neck, or left chest where pulses can readily be received. In another example where body temperature of the patient 112 is also to be detected, another bio monitoring patch 110 is placed at an armpit of the patient 112. In these examples, multiple bio monitoring patches 110 may be configured to act as a single bio monitoring patch 110. For example, the detected and stored body temperate at the armpit is transmitted and stored at the bio monitoring patch 110 in the wrist of the patient 112.

At block 504, determining if the bio monitoring patch is used to detect vital signs of the person is performed. If YES, then following YES signal at block 506, receiving electrical signals from multiple stimulators, stimulator contacts, or sensors is performed. For example, the bio monitoring patch 110 contains the stimulators, stimulator contacts, or sensors (e.g., 202) that converts the body temperature of the patient 112 into electrical signals. In this example, the electrical signals may represent an electrical equivalence of measured body temperature of the patient 112. In another example, the stimulators, stimulator contacts, or sensors 202 may be a transducer that converts acoustic signals from heart organ pulses to electrical signals. In this example, the electrical signals may represent beating frequency of the heart organ of the patient 112. In these examples, the electrical signals are received by a SOC microprocessor (e.g., SOC microprocessor 204).

At block 508, storing the electrical signals is performed. For example, the electrical signals that are measured at different time intervals by each of the stimulators, stimulator contacts, or sensors 202 are stored in a storage device (e.g., NVM 206). The stored electrical signals may be utilized as variables in implementing software program that may be configured at the SOC microprocessor 204.

At block 510, configuring the SOC microprocessor to execute the software program that utilizes the stored electrical signals is performed. For example, the SOC microprocessor 204 is configured to process and provide a summary of the stored electrical signals (e.g., body temperature) within the last twenty four hours. In this example, the SOC microprocessor 204 utilizes the stored electrical signals for patient's body temperature in the last twenty four hours as variables to generate the summary of the stored electrical signals.

In another example, the SOC microprocessor 204 is configured to process the stored electrical signals for both the body temperature and heartbeat rate in the last two hours and the SOC microprocessor 204 transmits an emergency signal if a certain threshold is reached. In this example, the SOC microprocessor 204 utilizes the body temperature and the heartbeat rate at the same time as variables to determine if the threshold is reached.

At block 512, transmitting an output of the SOC microprocessor 204 is performed. For example, if the SOC microprocessor 204 is configured to execute the software program and an output produced by this implementation includes transmission of the emergency signal to a wireless device (e.g., wireless device 104), then the bio monitoring patch 110 transmits the emergency signal through a network (e.g., network 114) or other radio signals (e.g., cellular signal, Wi-Fi signal, Bluetooth™ signal, etc.).

In an implementation, the wireless device 104 is configured to integrate different electrical signals that may be received from the bio monitoring patches 110. For example, if three separate bio monitoring patches 110 are attached to different body parts of the patient 112, then the wireless device 104 may be configured to process and utilize the electrical signals from the three separate bio monitoring patches 110 as variables to software program that may be implemented in the wireless device 104. In other words, the wireless device 104 may be configured to control operations of the bio monitoring patches 110 through the network 114 or other radio signals.

With reference to block 504, if the bio monitoring patch 110 is not utilized as a detector of vital signs, then following the NO signal at block 514, the bio monitoring patch 110 is configured to receive a wireless triggering signal. For example, the bio monitoring patch 110 receives the wireless triggering signal through its antenna (e.g., antenna 208). In this example, the wireless triggering signal may include a request signal from the wireless device 104 to the bio monitoring patch 110 to perform a specific operation.

At block 516, processing the wireless triggering signal is performed. For example the SOC microprocessor 204 is configured to process the received wireless triggering signal. In this example, the SOC microprocessor 204 may first determine authenticity of the wireless triggering signal and then utilizes the wireless triggering signal according to a configured program in the SOC microprocessor 204. The configured program may, for example, include delivering electrical pulses to muscles of the patient 112, changing frequency of detecting the vital signs, clearing memory contents at the NVM 206, and the like.

At block 518, performing an operation according to the processed wireless triggering signal is performed. For example, the operation may include the delivering of electrical pulses to the muscles of the patient 112. In this example, the stimulators, stimulator contacts, or sensors 202 may be configured to deliver the electrical pulses to the muscles of the patient 112.

What is claimed is:

1. A system on chip (SOC) device comprising:
at least one stimulator, stimulator contact, or sensor configured to convert a vital sign of a user into electrical signal;
memory that is configured to store the electrical signal;
a SOC microprocessor unit that is configured to process and utilize the stored electrical signal in implementing a configured software program in the SOC microprocessor, the configured software program combines the stored electrical signal to produce an output signal; and
an antenna that is configured to transmit the output signal of the SOC microprocessor.

2. The SOC device as recited in claim 1, wherein the at least one stimulator, stimulator contact, or sensor measures the vital sign that includes one or more of the following: a heartbeat rate, blood sugar, oxygen, blood pressure, muscle activity, neurological activity, brain waves, or body temperature.

3. The SOC device as recited in claim 1, wherein the at least one stimulator, stimulator contact, or sensor is configured to deliver electrical pulses.

4. The SOC device as recited in claim 1, wherein the memory is a non-volatile memory (NVM) that stores the electrical signals that are measured at different time intervals, the stored electrical signals are utilized as variables in implementing the configured software program at the SOC microprocessor.

5. The SOC device as recited in claim 1, wherein SOC microprocessor implements the software program that includes deriving a summary of the stored electrical signals, changing periods of vital signs detection, or clearing contents of non-volatile memory (NVM).

6. The SOC device as recited in claim 1, wherein the SOC microprocessor is configured to control another SOC device.

7. The SOC device as recited in claim 1, wherein the antenna is configured to receive a triggering signal from a wireless device, the triggering signal includes a request signal to the SOC device to perform an operation.

8. The SOC device as recited in claim 1, wherein the antenna is a near field communications (NFC) antenna.

9. A wireless device comprising: the SOC device as recited in claim

10. A wireless device comprising:
a memory;
a processor coupled to the memory, the processor includes one or more processor-executable instructions that, when executed, perform operations comprising:
receiving data that includes vital sign measurements;
storing the vital sign measurements;
executing a software program that utilizes the stored vital sign measurements.

11. The wireless device as recited in claim 10, wherein the memory stores the vital sign measurements that includes measurements of vital signs at different time intervals.

12. The wireless device as recited in claim 10, wherein the vital sign measurements include a heartbeat rate, blood sugar, oxygen, blood pressure, muscle activity, neurological activity, brain waves, or body temperature.

13. The wireless device as recited in claim 10, wherein the software program is configured to provide a triggering signal that is transmitted to a bandage patch configured to enable transmission of the vital sign measurements to the wireless device.

14. The wireless device as recited in claim 10, wherein the software program is configured to control operations of a bandage patch configured to detect vital measurements of a person.

15. The wireless device as recited in claim 10, further comprising an antenna configured to receive the data through a network or a radio signal.

16. The wireless device as recited in claim 10, further comprising a chip set that is configured to provide intercommunications between the memory and the processor.

17. A method of wireless electronic monitoring by a system on chip (SOC) device, the method comprising:
detecting vital signs from a body of a person, the vital signs are converted into electrical signal;
receiving the electrical signal;
storing the electrical signal;
configuring a microprocessor to execute software program that utilizes the stored electrical signal; and
transmitting an output of the microprocessor.

18. The method as recited in claim 17, the vital signs include at least one of a heartbeat rate, blood sugar, oxygen, blood pressure, muscle activity, neurological activity, brain waves, or body temperature.

19. The method as recited in claim 17, wherein storing the electrical signal includes measuring at different time intervals, the stored electrical signals are utilized as variables in implementing the configured software program at the microprocessor.

20. The method as recited in claim 17, wherein microprocessor implements the software program that includes deriving a summary of the stored electrical signals, changing frequency periods of vital signs detection, or clearing contents of non-volatile memory.

21. The method as recited in claim 17, wherein the transmitting the output includes delivering medicine, electrical, optical, radio frequency and/or mechanical energy through a stimulator, stimulator contact, and/or sensor.
